SEQUENCE LISTING (11 sequences)

SEQ ID NO: 1 - DNA, 978 bp, Artificial Sequence
  Description of Artificial Sequence cDNA of CMSP 0
atgagaactcaattgcttattgctgctgctttaggtttaaccttattaggtttaacttcctatttattcctccacaagtctactcaagttggatacactgatgactaaattaacatgtggaagggcttcaagaagacctacaacaaaaaattctcttctgaagatgctgactaagaagcttacagaatgaacgtcttcttcgataacgttgaatacgcttcataagattctaccatgggtattaccaagtttatggaccttacccctgttgaatttgcctaactttacttgaatcccattgaaaacgttgaaggttctattgaaactttctaagctattcaagctaatggagatattgttgtcgattgggttgctaagggtgctgtcacacctgttaaggatcaaggtggttgtggtggttgttggtctttcgctactactggtggtgttgaaggtgctaactttgtctacaaaaatgtcctccctaacttatctgaacaataattaatcgactgtgacacttaaaacagtggttgcggtggtggtttaagagacgttgccttaaactacgttaaggcaactggtttggccactgaataagattatccttatgaagctaaggatggtaagtgcagacttgaaggtaagagccacccttggactgtttctggttacacttctattaagtaatgcgctgacctcgttactgctattcaaaaggcccctgttaccgttggtatcgatgcttctaacctctaattctacactggtggtatcttttctaagtgcgccactaacatcaaccacggtgttttacttgttggttacgactctgttaatcaatcttggaaggtcaagaactcctggggtcctaacttcggtgaacacggttacttctaactctctgctaaggttactggtgaccaaattgctaacacttgcggtatttgctctagagcttatgctccttacatttga

SEQ ID NO: 2 - protein, 313 aa, Tetrahymena thermophila
MRTQLLIAAALGLTLLGLTSYLFLHKSTQVGYTDDQINMWKGFKKTYNKKFSSEDADQEAYRMNVFFDNVEYASQDSTMGITKFMDLTPVEFAQLYLNPIENVEGSIETFQAIQANGDIVVDWVAKGAVTPVKDQGGCGGCWSFATGGVEGANFVYKNVPNLSEQQLIDCDTQSGCGGGLRDVALNYKATGLATEQDYPYEKDGKCRLEGKSHPWVSGYTSIKQCADLVAIQKAPVTVGIDASLQFYTGGIFSKCATINHGVLLVGYDSVNSWKVKNSWGPNFGEGYFQLSAKVTGDQINTCGICSRAYAPYI

SEQ ID NO: 3 - DNA, 1112 bp, Artificial Sequence
  Description of Artificial Sequence 5′ non-translated region (position -370 to -1) of the CMSP 0 gene
aaaaaagtttacccattgttccgattttatatttaaaaattaaagaaagaattaaattgaatctttttttcttttataaaatctataaaagattgagataacaaaaagttgataaaaatataaaatattcatacatttaatttaagcatattaaaatacatttcatagttgaaaaataaagaaaacagtatctataaaaactatgctgaaagtttaatgctgaaagtttacttctcgtttttatttaacttttctagtttaaaataattattaatatgaattgaaataattgttactattagcttttattgaattcaatttattaaaaattggatctaatgtaatcttagaaataataactaaaaatgggatttgaaaaatctagataagaatataaaattaaaaatcaaattaatcgaatcttttattacatctcattaaaaagttaataaaataaaaaataaagttttattcttattttaatatcattttttaaattaccataatcaattttaacttaaagcttcaataaaaaaatatatattttagaaactttaataaactattgagcaacttataaagaattaaaaatatttttcatttgtaaaatgaaatgaaaaatattatcctgagcttacaaatcttttataaacatttttttatttataattttcttttatattaaggttttctaatatcgaataagatttctgctcagagaaaattttctgcaatttaataaataataaaagaatttttatgtaaaagaattaatttaatctagaccttagaaaatgaatgaatcaatatatacttttaactctgttttgcatgagtaaacaaatgagttttttacatcaaaacagtttttacattttgtatgtaatcaaaaaagctttactttgactaaaatattgaaagatttgctttaaagctaaaatatacattaaactattaaagatttttatttataattctttcataataaatcattaacaaataaacaaacaaacaaagaaaaaaattatttagtcaagctttaaaaaattattaattgaaataatatttgatataattaaattaatctaaaaaacataagatagaataaaata

SEQ ID NO: 4 - DNA, 343 bp, Artificial Sequence
  Description of Artificial Sequence non-translated terminator region (position 979 to 1321) of the CMSP 0 gene
aaaatctttcaaaaatatataaaatgattaatataaaactttatatttttatagtaattaatattaaaaactttttgtttaactatttataggaaataatattatttactataaacaagccagacaactattcttttgtttttattacttttttatacaaaaaactgataacaaatcaattcaaataaatattctattatcatgaaagtcttacattttattttaacaaaaaataaaaagaatctattttttaattttgacttgaattacaacttttataacaaatcaaactttaacaatttataattaaaaattttaatccactaattaattgactgaatat

SEQ ID NO: 5 - protein, 116 aa, Artificial Sequence
  Description of Artificial Sequence amino acid sequence of the Pre/pro-peptide (amino acids 1 to 116) of CMSP 0
MRTQLLIAAALGLTLLGLTSYLFLHKSTQVGYTDDQINMWKGFKKTYNKKFSSEDADQEAYRMNVFFDNVEYASQDSTMGITKFMDLTPVEFAQLYLNPIENVEGSIETFQAIQAN

SEQ ID NO: 6 - protein, 197 aa, Artificial Sequence
  Description of Artificial Sequence amino acid sequence of the mature protein CMSP 0 (amino acids 117 to 325)
GDIVVDWVAKGAVTPVKDQGGCGGCWSFATGGVEGANFVYKNVPNLSEQQLIDCDTQSGCGGGLRDVALNYKATGLATEQDYPYEKDGKCRLEGKSHPWVSGYTSIKQCADLVAIQKAPVTVGIDASLQFYTGGIFSKCATINHGVLLVGYDSVNSWKVKNSWGPNFGEGYFQLSAKVTGDQINTCGICSRAYAPYI

SEQ ID NO: 7 - DNA, 1417 bp, Artificial Sequence
  Description of Artificial Sequence   DNA coding for 5′ and 3′ non-coding regions of the CMSP 1 gene
cttaacagagagtatcctgtaaattaaaagttattaacattccccactcgatcaatttcttttacactctttaaacgaaatgtttcgattttatccatcaaaattcttaattcttatatatttttaactattaaaatatcagattgataattcatgccatttgtggtgttttaaatacaataatacgtatatcatcttgcataactttcaaagccacaaaaactaattgatcctatttttatagattaggaaactatttcttttatattttgtagacatctaattattactttaaatactagattatctaatacatcctttcataaaagattcaattaaataaatttaaactaaacaaaaaaaatatgagctaaaaaattactgttactcttgttgctatcgctgctattgccgctatcactgctgctggcatttactactagaaccactaagctagccaattagaaaagtctttcaagagaaataccatccttgaataatggaacgaattcaagcaaaagtttggtaagaaatatgctgactaagaattcgaaagatacagaatcggagttttcgctcaaaatttagaagttatcaagaacgatccttccttcggtgttaccaagttcatggatatgaccccataagaattcgaacaatcctacttatctctctaactccaacaaaacttcaatgctgaaaaggttgatggtgactttaatggtgatattgattggacttaaaagggtgctgttactcctgtcaaggactaaggttcttgcggttcttgctgggctttctctgctatcggagctgttgaatctgctttgatcttgaacggtgaagacaagaacatcaatttggctgaataagaattggtcgactgtgctactactcccaagtacgaaaatgaaggttgcaacggtggttggatggactctgctttcgactacattattgatgaaaagatctcttaaaccaaggactacaagtacactgctagagacggcaagtgcaaggatacctcatcttttgaaaagaagtctatctctggatacaaggacattcctcaaggtgactgcaagtctctcttaaacgctctttcctaataacccgttgctattgcagttgatgcttcttcttggtaattctacaacaagggagttttatcatcctgtggcagcagacttaatcacggtgttttattaactggttacgttaacgaaacttacaaggttaagaactcttggggtacttcttggggtgaaaagggtttcatctaattaaagtcaggtaactcttgtggtctctgcaatgctgcctcttaccctcttgcttgaaaaaaatacttaaataattaaaaaaaatagtattattatataatccatattaaagtctttttttataaatctttaaa

SEQ ID NO: 8 - protein, 119 aa, Artificial Sequence
  Description of Artificial Sequence amino acid sequence of the pre/pro-peptide (amino acids 120 to 324) of the CMSP 1 protein
MSQKITVTLVAIAAIAAITAAGIYYQNHQASQLEKSFKRNTILEQWNEFKQKFGKKYADQEFERYRIGVFAQNLEVIKNDPSFGVTKFMDMTPQEFEQSYLSLQLQQNFNAEKVDGDFN

SEQ ID NO: 9 - protein, 205 aa, Tetrahymena thermophila
GDIDWTQKGAVTPVKDQGSCGSCWAFSAIGAVESALILNGEDKNINLAEQELVDCATTPKYENEGCNGGWMDSAFDYIIDEKISQTKDYKYTARDGKCKDTSSFEKKSISGYKDIPQGDCKSLLNALSQQPVAIAVDASSWQFYNKGVLSSCGSRLNHGVLLTGYVNETYKVKNSWGTSWGEKGFIQLKSGNSCGLCNAASYPLA

SEQ ID NO: 10 - DNA, 365 bp, Artificial Sequence
  Description of Artificial Sequence 5′non-translated promotor region of the CMSP 1 gene
cttaacagagagtatcctgtaaattaaaagttattaacattccccactcgatcaatttcttttacactctttaaacgaaatgtttcgattttatccatcaaaattcttaattcttatatatttttaactattaaaatatcagattgataattcatgccatttgtggtgttttaaatacaataatacgtatatcatcttgcataactttcaaagccacaaaaactaattgatcctatttttatagattaggaaactatttcttttatattttgtagacatctaattattactttaaatactagattatctaatacatcctttcataaaagattcaattaaataaatttaaactaaacaaaaaaaat

SEQ ID NO: 11 - DNA, 2433 bp, Artificial Sequence
  Description of Artificial Sequence nucleic acid sequence of CMSP 0 of Tetrahymena
aaaaaagtttacccattgttccgattttatatttaaaaattaaagaaagaattaaattgaatctttttttcttttataaaatctataaaagattgagataacaaaaagttgataaaaatataaaatattcatacatttaatttaagcatattaaaatacatttcatagttgaaaaataaagaaaacagtatctataaaaactatgctgaaagtttaatgctgaaagtttacttctcgtttttatttaacttttctagtttaaaataattattaatatgaattgaaataattgttactattagcttttattgaattcaatttattaaaaattggatctaatgtaatcttagaaataataactaaaaatgggatttgaaaaatctagataagaatataaaattaaaaatcaaattaatcgaatcttttattacatctcattaaaaagttaataaaataaaaaataaagttttattcttattttaatatcattttttaaattaccataatcaattttaacttaaagcttcaataaaaaaatatatattttagaaactttaataaactattgagcaacttataaagaattaaaaatatttttcatttgtaaaatgaaatgaaaaatattatcctgagcttacaaatcttttataaacatttttttatttataattttcttttatattaaggttttctaatatcgaataagatttctgctcagagaaaattttctgcaatttaataaataataaaagaatttttatgtaaaagaattaatttaatctagaccttagaaaatgaatgaatcaatatatacttttaactctgttttgcatgagtaaacaaatgagttttttacatcaaaacagtttttacattttgtatgtaatcaaaaaagctttactttgactaaaatattgaaagatttgctttaaagctaaaatatacattaaactattaaagatttttatttataattctttcataataaatcattaacaaataaacaaacaaacaaagaaaaaaattatttagtcaagctttaaaaaattattaattgaaataatatttgatataattaaattaatctaaaaaacataagatagaataaaataatgagaactcaattgcttattgctgctgctttaggtttaaccttattaggtttaacttcctatttattcctccacaagtctactcaagttggatacactgatgactaaattaacatgtggaagggcttcaagaagacctacaacaaaaaattctcttctgaagatgctgactaagaagcttacagaatgaacgtcttcttcgataacgttgaatacgcttcataagattctaccatgggtattaccaagtttatggaccttacccctgttgaatttgcctaactttacttgaatcccattgaaaacgttgaaggttctattgaaactttctaagctattcaagctaatggagatattgttgtcgattgggttgctaagggtgctgtcacacctgttaaggatcaaggtggttgtggtggttgttggtctttcgctactactggtggtgttgaaggtgctaactttgtctacaaaaatgtcctccctaacttatctgaacaataattaatcgactgtgacacttaaaacagtggttgcggtggtggtttaagagacgttgccttaaactacgttaaggcaactggtttggccactgaataagattatccttatgaagctaaggatggtaagtgcagacttgaaggtaagagccacccttggactgtttctggttacacttctattaagtaatgcgctgacctcgttactgctattcaaaaggcccctgttaccgttggtatcgatgcttctaacctctaattctacactggtggtatcttttctaagtgcgccactaacatcaaccacggtgttttacttgttggttacgactctgttaatcaatcttggaaggtcaagaactcctggggtcctaacttcggtgaacacggttacttctaactctctgctaaggttactggtgaccaaattgctaacacttgcggtatttgctctagagcttatgctccttacatttgaaaaatctttcaaaaatatataaaatgattaatataaaactttatatttttatagtaattaatattaaaaactttttgtttaactatttataggaaataatattatttactataaacaagccagacaactattcttttgtttttattacttttttatacaaaaaactgataacaaatcaattcaaataaatattctattatcatgaaagtcttacattttattttaacaaaaaataaaaagaatctattttttaattttgacttgaattacaacttttataacaaatcaaactttaacaatttataattaaaaattttaatccactaattaattgactgaatat

(19) United States
(12) Patent Application Publication
(10) Pub. No.: US 2006/0127973 A1
(43) Pub. Date: Jun. 15, 2006

(54) Dna sequences of major secreted proteins from the ciliate tetrahymena and use thereof

(76) Inventors: Marcus Hartmann, Munster (DE); Nadine Wolf, Munster (DE)

Correspondence Address: JACOBSON HOLMAN PLLC, 400 SEVENTH STREET N.W., SUITE 600, WASHINGTON, DC, 20004, US

(21) Appl. No.: 10/507,908

(22) PCT Filed: Mar. 19, 2003
(86) PCT No.: PCT/EP03/02856

§ 371 Date: Jun. 13, 2005

Related U.S. Application Data
(60) Provisional application No. 60/365,173, filed on Mar. 19, 2002.
(60) Provisional application No. 60/394,663, filed on Jul. 10, 2002.

(30) Foreign Application Priority Data
Mar. 19, 2002 (EP) 02006151.1
Mar. 22, 2002 (EP) 02006455.6
Apr. 12, 2002 (EP) 02008343.2
Jul. 11, 2002 (DE) 102 31 365.2

(51) Int. Cl. C07K 14/195; C07H 21/04; C12P 21/06; C12N 1/21; C12N 15/74

(52) U.S. Cl. 435/69.1; 435/320.1; 435/252.3; 530/350; 536/23.7

(57) ABSTRACT

A regulatory element of a DNA for an efficient heterologous expression of proteins in Tetrahymena ssp which efficient heterologous expression is performed under control of promotors and/or terminators which are derived from in Tetrahymena ssp naturally occurring DNA comprising promotors and/or terminators and a coding region for proteins secreted ion a high level and the expression of proteins secreted on a high level is independent of the cell-cycle of Tetrahymena ssp.

Furthermore a method is disclosed for the heterologous expression of proteins from Tetrahymena using gene constructs made from regulatory elements selected from the group consisting of promoters or terminators from Tetrahymena and coding nucleic acid sequences of a protein to be expressed heterologously, said regulatory elements from Tetrahymena being obtainable by: two-dimensional gel electrophoretical separation and isolation of the proteins (CMSP) selected from the group according to table 1; determination of at least one partial amino acid sequence of the proteins; establishing the nucleic acid sequence of the proteins and therefrom establishing the gene which codes for these proteins; establishing the regulatory elements of the coding region of said proteins.

In the heterologous expression of foreign proteins, yeasts, bacteria and mammal cells are of great importance to the biotechnological preparation and production of recombinant active substances. Bacterial expression systems based on E. coli or B. subtilis are used for the production of recombinant peptides or proteins, such as insulin, interleukin-2, tissue plasminogen activator, proteases and lipases. In Gram-negative bacteria, the expression systems are based, for example, on the use of genetic elements, such as the lac operon or the tryptophan operon. The proteins foreign to the host are produced either into “inclusion bodies” within the cell, or when expression systems based on β-lactamase genes are used, into the periplasmic space. The production of recombinant proteins into the surrounding fermentation medium has not been established. In Gram-Positive bacteria, to date, almost exclusively cell-inherent proteins are introduced in expression systems and expressed.

Yeasts, such as S. cerevisiae, Hansenula polymorpha, Kluyveromyces lactis or Pichia pastoris or methanolica, are also employed for the heterologous expression of recombinant proteins, such as surface antigens, human factor XIIIa, bovine pro-chymosin, or phytase. In yeasts, the expression systems are based on shuttle vectors (vectors having both yeast and bacterial portions) which are based (depending on the yeast species) on the genetic elements of galacto-kinase-epimerase, methanol oxidase, acid phosphatase or alcohol-dehydrogenase. As a rule, the recombinant protein is produced into the cytoplasm of the cell. When yeast-inherent signal sequences, such as the alpha factor, are used, the expressed proteins may also be secreted into the fermentation medium. The glycosylation of secreted proteins is effected according to the “high mannose” type, and frequently there are hyperglycosylations on the protein which may result in the formation of antibodies in the patient.

In addition to yeasts and bacteria, mammal cells, such as various cell types from rodents (CHO cells, C127 cells), simians (vero, CV-1 or COS cells) or immortalized human cell lines (PER.C6), are primarily employed for heterologous expression.

Here, the expression systems are based on recombinant viruses (BPV vector, adenoviral vectors) or on shuttle vectors. To regulate the expression, viral SV40 enhancer/promoter systems or cellular enhancer elements are employed, inter alia. The recombinant proteins, such as erythropoietin, are secreted into the fermentation medium because the foreign genes usually bring their own signal sequences, which are understood by the expression system and used for targeting.

Further, insect cells, such as baculovirus systems, Drosophila S2 cells and Lepido-ptera cells, are employed for expression.

Further, for the biotechnological production of glycosylated extracellular enzymes, protozoans of the genus Tetrahymena are employed. Tetrahymena will grow on inexpensive fermentation media using standard fermentation methods. For the transformation of such Tetrahymena cells, vectors are available which are based on the rDNA elements of Tetrahymena. For the heterologous expression of bacterial proteins in Tetrahymena, DNA constructs made from genes from Tetrahymena are employed. When suitable genetic elements for the regulation of the transcription, targeting and glycosylation of foreign proteins are available, Tetrahymena is an ideal expression system for the inexpensive production of therapeutic recombinant proteins.

The Gram-negative bacterial expression systems used to date usually lead to the formation of “inclusion bodies” in the cell, accompanied by a denaturing of the proteins. To recover the recombinant protein, the cells must be lysed, and the denatured inactive protein must be folded back to function. This causes additional cost-intensive process steps and reduces the yield of the desired protein. Glycosylation, which is important to eukaryotic proteins, is completely omitted. When Gram-positive bacterial expression systems are used, degradation of the target protein due to high proteolytic activities in the fermentation broth is an additional problem.

When yeasts, such as Saccharomyces cerevisiae, are used for heterologous expression, the desired target protein is often produced only into the cell, from where it must be removed by cell lysis. As in bacterial expression systems, this causes additional time- and cost-intensive process steps. When yeast-inherent signal peptides are used, the foreign proteins are not correctly spliced and glycosylated for secretion. Especially the hyperglycosylation of the expressed proteins by S. cerevisiae results in the formation of antibodies in the human organism. In addition, the synthesized proteins are often degraded intracellularly. When other yeasts, such as Pichia pastoris, are used, the expression of the foreign genes must be induced by adding methanol to the fermentation medium because so-called AOX1 promoters are used. AOX1 promoters are induced by the addition of methanol. This is problematic on an industrial scale since methanol is a considerable safety risk on this scale because of its inflammability. When insect cell systems, such as the baculovirus system based on Sf9 cells, are employed, the introduction of foreign DNA is extremely complicated since recombinant baculovirus particles must first be produced in a complicated process. In addition, the transfection of the expressing cells is effected only in the production culture by large amounts of baculovirus particles (“high titer stocks”). Further, after the infection of the Sf9 cells by the baculoviruses, lysis of these cells occurs, which results in the contamination of the culture supernatant with intracellular proteins. Therefore, a stable expression is not possible in these cell lines. Other insect cell systems, such as the Drosophila S2 cell system, grow very slowly (more slowly than the mammal cells stated below) and exhibit comparably low expression rates.

In contrast, when mammal cell systems are employed for the production of recombinant proteins, the desired proteins are found in the fermentation medium in an extracellular state, correctly spliced and glycosylated. However, what is disadvantageous here is, on the one hand, the low expression rate due to the defective processing and inefficient translation of genes which have been introduced into the genome of the production cell line via viral vectors. On the other hand, the serum-containing fermentation media for mammal cells are extremely cost-intensive. In addition, the fermentation technology for the shear-sensitive cell lines is complicated and similarly expensive due to constructions for bubble-free aeration. Further problems arise from the high infection risk for the cell lines from mycoplasmas and viruses. All in all, the use of mammal cells for the biotechnological preparation of recombinant proteins results in very high costs, safety demands and low yields. The safety problems have been solved in part by the use of immortalized human cell lines (e.g., PER.C6). However, these cell lines also have the disadvantages of mammal cell technology. In addition to the above mentioned drawbacks, these include the necessity of adding CO₂ gas and the time-consuming experimental procedure for transformation/transfection by dual vector systems.

To the use of ciliates, such as Tetrahymena, the above mentioned drawbacks in the production of recombinant proteins do not apply. Thus, for example, some acid hydrolases which are involved in the digestion of food particles are exported from the cell in high quantities and with complex glycosylation.

In J. Euk. Microbiol. 43 (4), 1996, pages 295 to 303, Alam et al. describe the cloning of a gene which codes for the acid α-glucosidase of Tetrahymena pyriformis. However, only a small portion of the protein is exported from the cell. Further, the International Patent Application PCT/EP 00/01853 describes the gene of a β-hexosaminidase from Tetrahymena thermophila which is known, however, to be exported from the cell to only about 80%. The gene of β-hexosaminidase claimed in this patent includes the nucleotide sequence which codes for the pre/pro peptide of this enzyme. As mentioned above, the enzyme β-hexosaminidase is secreted into the surrounding culture medium to only about 80%. About 20% of the enzyme is targeted into the cytoplasm membrane and can be localized there. For this reason, pre/pro peptides of β-hexosaminidase, when positioned in front of a protein foreign to the host by genetic engineering methods, will target only about 20% of the protein foreign to the host into the cytoplasma membrane on the surface of Tetrahymena thermophila. This is associated with a considerable process-technological disadvantage for the production of recombinant active substances. On the one hand, the yield is decreased because part of the expressed protein remains in the cells bound to the membrane, and it is not possible to purify the entire expressed protein from the fermenter broth. On the other hand, the protein foreign to the host in the cell membrane can exert toxic effects on the host cells and thus slow down the cell growth. PCT/EP 02/00578 discloses the gene of a phospholipase A₁ (PLA₁) from Tetrahymena thermophila. This enzyme is released exclusively into the surrounding fermentation medium so that, when pre-pro sequences of the PLA₁ are used for the heterologous expression of a recombinant active substance, the latter can be found exclusively in the surrounding culture medium.

All proteins which are described in the above mentioned documents belong to the acid hydrolases and are also referred to as extracellular lysosomal enzymes in the technical literature. However, these enzymes in each case comprise only a small proportion of the proteins continuously secreted by Tetrahymena. Thus, under defined conditions, β-hexosaminidase represents about 0.1% of the total amount of protein secreted. Under defined conditions, the PLA1 represents about 0.5% of the total amount of protein secreted. From the proportion of the total amount of proteins of the above mentioned secreted enzymes, it can be seen that the regulatory elements (promoters, pre/pro peptides, terminators) of these enzymes cause only a low constitutive expression and secretion into the surrounding culture medium. However, for the expression of recombinant active substances in Tetrahymena, a high constitutive expression and secretion of the proteins is required for enhancing the productivity. This in turn requires strong promoters, pre/pro sequences and terminators. Although genes which have relatively strong promoters and terminators, such as histones or tubulins, are known in Tetrahymena, these promoters are dependent on the cell cycle and therefore are active only in cultures under logarithmic growth. Therefore, such regulatory elements which are dependent on the cell cycle are not suitable for maintaining the expression of recombinant active substances in a steady-state culture.

Thus, the problems involved in the previously available regulatory elements on the gene level for the expression of foreign proteins in Tetrahymena are as follows:

The available sequences of acid hydrolases contain regulatory sequences which do not result in a high expression and secretion of the foreign protein. On the other hand, the available strong promoters are dependent on the cell cycle and are not suitable for expression during the long steady-state growth phases of cultures.

It is an object of the invention to provide proteins from Tetrahymena and the DNA sequences derived therefrom. The DNA is to enable heterologous proteins in an expression system to be exported into the fermentation medium after expression in Tetrahymena. Further, DNA sequences are to be provided which contain regulatory elements that cause a constitutive, i.e., independent of the cell cycle, transcription of the downstream genes of heterologous proteins. Constitutive transcription has the advantage that the heterologously expressed proteins are constantly under expression in the host organism without being affected by the cell cycle. Thus, even during a steady-state growth phase with low cellular growth, transcription of the foreign gene can be effected and the heterologous protein can undergo expression.

The object of the invention is achieved by a regulatory element of a DNA for an efficient heterologous expression of proteins in Tetrahymena ssp which efficient heterologous expression is performed under control of promoters and/or terminators which are derived from in Tetrahymena ssp naturally occurring DNA comprising promotors and/or terminators and a coding region for proteins secreted on a high level and the expression of proteins secreted on a high level is independent of the cell-cycle of Tetrahymena ssp.

The term “regulatory element” means in particular any part of a nucleic acid which regulates, influences or controls the expression of a gene.

The term “heterologous expression” is well known to the person skilled in the art.

The term “efficient heterologous expression” means an expression of the protein which is secreted into a medium about 2 to 5 fold stronger than the protein called phospholipase A₁.

The term “protein secreted on a high level” or its grammatical equivalents means a secretion of the protein into a fermentation broth without significant loss of protein on the way from the ribosome to extra cellular space in particular the fermentation broth.

Expression of proteins indepently of the “cell-cycle of Tetrahymena ssp” and its grammatical equivalents means typically a constitutive expression of proteins.

In a specific embodiment of the invention the regulatory element of a DNA of the invnetion is obtainable by
isolating proteins of Tetrahymena ssp which proteins are secreted on a high level into a fermentation broth and
the secretion of the proteins is occurring on a high level independently of the cell-cycle of Tetrahymena ssp,
determination of at least one partial amino acid sequence of the proteins;
establishing the nucleic acid sequence of the proteins and there from establishing the gene which codes for these proteins;
establishing the regulatory elements of the coding region of said proteins by methods known as such in molecular biology.

The regulatory element of the invention is in particular obtainable from Tetrahymena ssp using gene constructs made from regulatory elements selected from the group consisting of promoters or terminators from Tetrahymena and coding nucleic acid sequences of a protein to be expressed heterologously, said regulatory elements from Tetrahymena being obtainable by:

two-dimensional gel electrophoretical separation and isolation of the proteins (CMSP) selected from the group consisting of:

| | | Isoelectric |
|---|---|---|
| | Molecular weight | point |
| | ±0.5 kD | ±0.2 units |
| CMSP No. | (kD) | (pH units) |
| 0 | 22.22 | 5.9 |
| 1 | 24.96 | 7.5 |
| 2 | 16.04 | 7.3 |
| 3 | 24.96 | 6.8 |
| 4 | 23.76 | 6.5 |
| 5 | 23.76 | 5.5 |
| 6 | 30.38 | 6.8 |
| 7 | 31.91 | 6.8 |
| 8 | 25 | 8.5 |
| 9 | 16.44 | 6.9 |
| 10 | 11.2 | 5.5 |
| 11 | 37.9 | 7.2 |
| 12 | 37.9 | 7.8 |
| 13 | 21.54 | 7.4 |
| 14 | 24.36 | 8.7 |
| 15 | 14.4 | 4.7 |
| 16 | 14.4 | 4.9 |
| 17 | 19.05 | 6.4 |
| 18 | 22.6 | 7.2 |
| 19 | 20.01 | 7.4 |
| 20 | 29.6 | 7.3 |
| 21 | 30 | 7.3 |
| 22 | 30.38 | 7.5 |

determination of at least one partial amino acid sequence of the proteins;
establishing the nucleic acid sequence of the proteins and therefrom establishing the gene which codes for these proteins;
establishing the regulatory elements of the coding region of said proteins.

In a particular embodiment of the present invention the “regulatory element” is
a) a promotor region in the 5′ up-stream sequence of the nucleic acid called CMSP 0 (Seq. ID. No. 3) with tata-boxes (−140 to −143, −300 to −303, −445 to −448 and −570 to −575) and caat boxes (−305 to −308 and −602 to −605).
b) A terminator region the 3′ down-stream sequence of the nucleic acid calles CMSP 0 (also Seq. ID. No. 4) with a region from +979 to +1321. and
c) a promotor region in the 5′ up-stream sequence of the nucleic acid called CMSP 1 (Seq. ID. No. 10) with tata-boxes (−99 to −102, −123 to −126 and −248 to −251) and caat boxes (−30 to −33 and −310 to −313).

Subject matter of the invention is also a method for heterologous expression of proteins in Tetrahymena ssp in a broth which proteins are secreted on a high level into the broth by employing a regulatory element of one of the invention.

In one embodiment of the invention the method for the heterologous expression of proteins from Tetrahymena is using gene constructions made from regulatory elements selected from the group consisting of promoters or terminators from Tetrahymena and coding nucleic acid sequences of a protein to be expressed heterologously, said regulatory elements from Tetrahymena being obtainable by isolating the proteins (CMSP) selected from the group consisting of proteins of Table 1, determination of at least one partial amino acid sequence of the proteins, establishing therefrom the nucleic acid sequence of the proteins and establishing the gene coding for these proteins, and establishing the regulatory elements of the coding region of said proteins.

The proteins stated in Table 1 were separated in a two-dimensional gel electrophoresis and identified. Tetrahymena releases a wide variety of further proteins into the surrounding culture medium. The proteins according to Table 1 are exported from the cell in very large amounts and occur in the surrounding culture medium in a much higher concentration as compared with the known acid hydrolases of Tetrahymena. In the following, they are referred to as ciliate major secreted proteins (CMSPs). By means of denaturing two-dimensional gel electrophoresis, it could be detected that the major secreted proteins occur in the cell-free supernatant of a Tetrahymena culture in a significantly higher concentration as compared with the previously described acid hydrolases α-glucosidase, β-hexosaminidase and PLA₁. Table 1 shows a listing of the ciliate major secreted proteins (CMSPs) which are biochemically characterized by their molecular weight and their isolelectric point.

TABLE 1
Biochemical characterization of the ciliate major secreted proteins
(CMSP 0 to 22).

| | Molecular | |
|---|---|---|
| | weight | Isoelectric point |
| | ±0.5 kD | ±0.2 units |
| CMSP No. | (kD) | (pH units) |
| 0 | 22.22 | 5.9 |
| 1 | 24.96 | 7.5 |
| 2 | 16.04 | 7.3 |
| 3 | 24.96 | 6.8 |
| 4 | 23.76 | 6.5 |
| 5 | 23.76 | 5.5 |
| 6 | 30.38 | 6.8 |
| 7 | 31.91 | 6.8 |
| 8 | 25 | 8.5 |
| 9 | 16.44 | 6.9 |
| 10 | 11.2 | 5.5 |
| 11 | 37.9 | 7.2 |
| 12 | 37.9 | 7.8 |
| 13 | 21.54 | 7.4 |
| 14 | 24.36 | 8.7 |
| 15 | 14.4 | 4.7 |
| 16 | 14.4 | 4.9 |
| 17 | 19.05 | 6.4 |
| 18 | 22.6 | 7.2 |
| 19 | 20.01 | 7.4 |
| 20 | 29.6 | 7.3 |
| 21 | 30 | 7.3 |
| 22 | 30.38 | 7.5 |

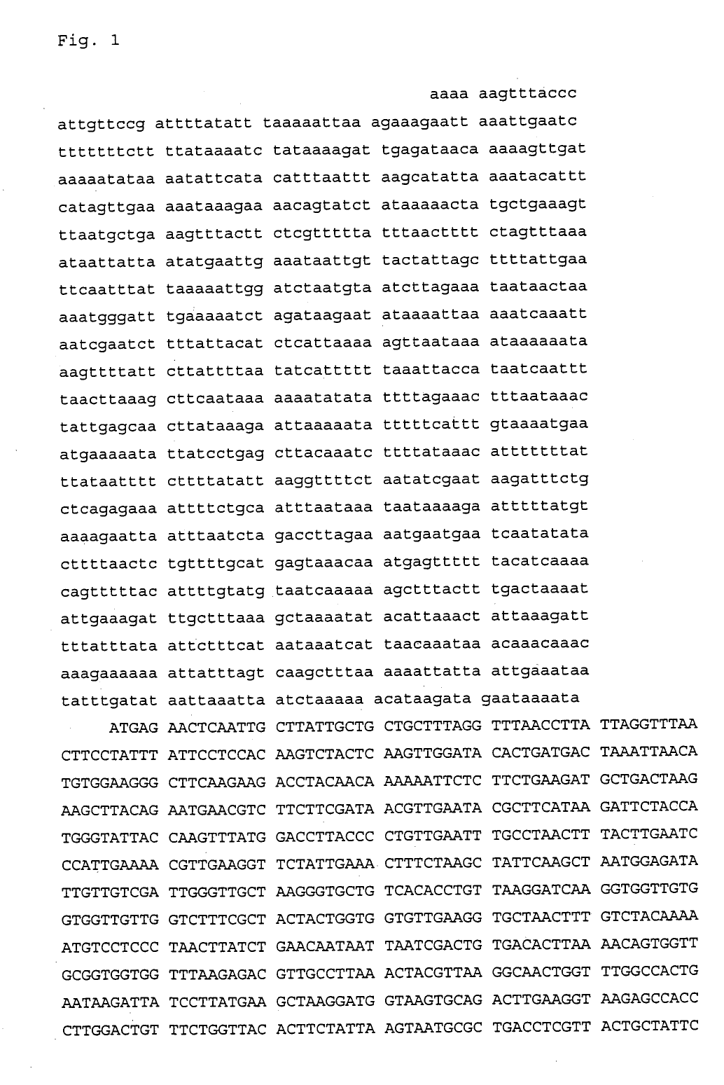

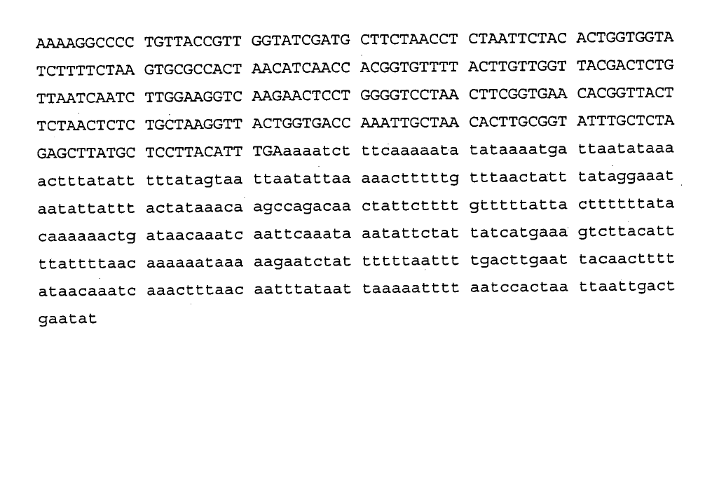

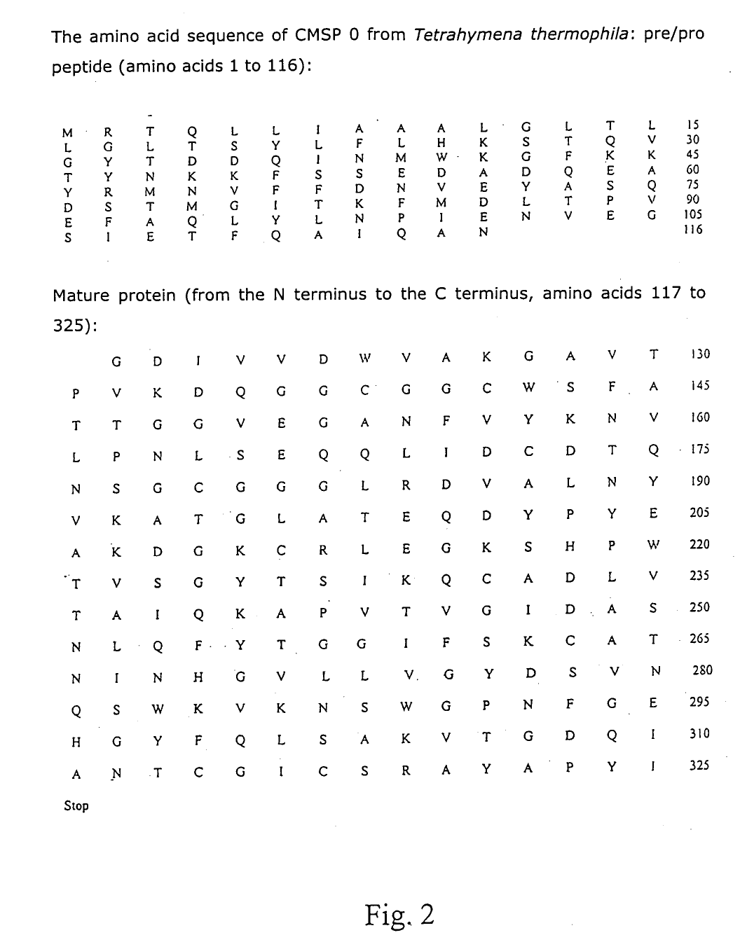

One example of a gene of a ciliate major secreted protein (CMSP) is given by the nucleotide sequence of CMSP 0 in FIG. 1. From this, as an example of a ciliate major secreted protein (CMSP), the amino acid sequence (SEQ ID NO: 2) of CMSP 0 in FIG. 2 results.

FIG. 1: The nucleic acid sequence of CMSP 0 from Tetrahymena thermophila:

The nucleic acid sequence Seq. ID. No. 3 of the non-translated region (upstream region) upstream from the coding sequence region of CMSP 0 from Tetrahymena is found between position −370 and position −1 (represented in lowercase letters). The coding sequence region of the cDNA (Seq. ID. No. 1) is represented in capital letters. With the start codon ATG, the numbering of the sequence begins. Regions known from the protein sequencing are printed in boldface, and the stop codon is underlined. The mature protein Seq. ID. No. 6 is coded from base 349. The sequence protocol from base 1 to base 348 represents the pre/pro sequence of CMSP 0 (Seq. ID. No. 5). The sequence protocol from base 349 to base 978 represents the sequence of the mature protein. In position 976, there is the translation stop TGA. The nucleic acid sequence (Seq. ID. No. 4) from position 979 to position 1321, which is below the coding sequence of the protein CMSP 0 from Tetrahymena, is the downstream region of the protein, which is not translated (also represented in lowercase letters).

The invention relates in particular to proteins having the Seq. ID. No. 2 and a nucleic acid coding for them of Seq. ID. No. 1.

The DNA sequences of the major secreted proteins according to the invention include an upstream region which bears the promoter elements for the initiation of transcription, a signal peptide and a pro-peptide, further genetic elements for the targeting of proteins and a downstream region which contains genetic elements for the termination of transcription. The use of these sequences in a vector enables the expression of heterologously expressed proteins independently of the cell cycle and to transport in large amounts them out of the cell and into the surrounding culture medium.

FIG. 1 shows a nucleic acid coding for the upstream region, the coding region and the downstream region of the major secreted protein 2 from Tetrahymena (CMSP 0).

FIG. 2 shows the amino acid sequence of the pre/pro peptide of CMSP 0 from Tetrahymena thermophila.

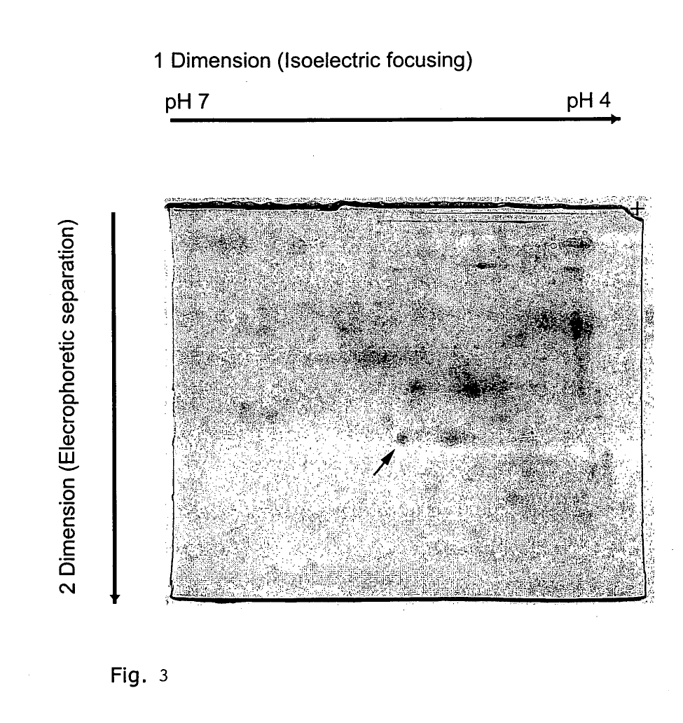

FIG. 3 it is shown that CMSP-Proteins of the invention are generally stronger secreted than the protein called phospholipase A₁ (PLA₁).

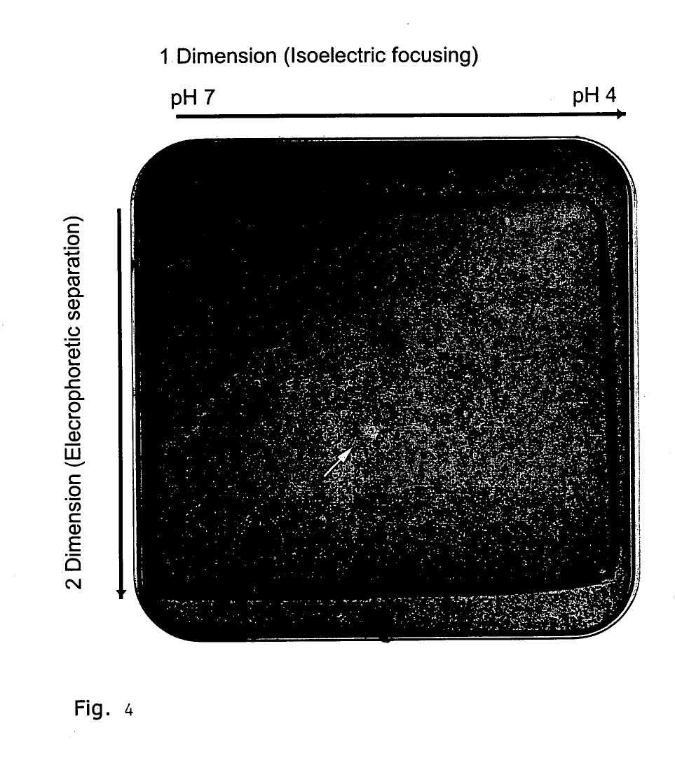

FIG. 4 shows a native two dimensional gelelectrophoresis of concentrated supernatants from a Tetrahymena thermophila culture. The protein PLA₁ is marked with black arrow. A corresponding lecithin-agarose overlay shows the corresponding lytic halo, which is a result of the enzymatic acitivity of the transfered PLA₁ (white arrow). The intensity of the other stained proteins spots on the stained gel, which are the CMSP's, shows that these proteins are much more abundant than the PLA₁-spot. This result shows that the CMSP-proteins are at about 2 to 5 fold stronger expressed than the PLA1.

The invention also relates to the regulatory elements, especially the promoter and terminator regions of the genes of the proteins according to the invention. In particular, these are the nucleic acids in the region from −370 to −1. In addition, the invention relates, in particular, to the pre/pro peptides of the proteins according to the invention. In particular, these are the amino acids 1 to 116 of the major secreted protein CMSP 0 according to the invention.

A further aspect of the invention is the use of the nucleic acid sequences of major secreted proteins from ciliates according to the invention or parts thereof for the homologous or heterologous expression of recombinant proteins and peptides, and for homologous or heterologous recombination (“knock-out, “gene replacement”).

The invention also relates to a method in which the nucleic acids or parts thereof according to the invention which code for CMSPs are combined with the usual, in homologous or heterologous expression, enhancers, such as the NF-1 region (a cytomegalovirus enhancer), promoters, such as the lac, trc, tic or tac promoters, the promoters of classes II and III of the T7 RNAP system, bacteriophage T7 and SP6 promoters, aprE, amylase or spac promoters for Bacillus expression systems, AOX1, AUG1 and 2 or GAPP promoters (Pichia) for yeast expression systems, RSV promoter (SV40 virus), CMV promoter (Cytomegalovirus), AFP promoter (adenoviruses) or metallothionine promoters for mammal expression systems, Sindbis virus promoters or Semlike forest virus promoters for insect cells, promoters for insect cell expression systems, such as hsp70, DS47, actin 5C or copia, plant-specific promoters, such as 35S promoter (cauliflower mosaic virus), amylase promoter or class I patatin promoter, operators, such as the tet operator, signal peptides, such as a-MF prepro signal sequences (Saccharomyces), origins, terminators, antibiotic and drug resistances, such as ampicillin, kanamycin, streptomycin, chloramphenicol, penicillin, amphotericin, cycloheximide, 6-methylpurine, paromomycin, hygromycin, α-amanatin, auxotrophy markers, such as the gene of dihydrofolate reductase, or other nucleic acids or DNA fragments, or all kinds of sequences from viroids, viruses, bacteria, archezoans, protozoans, fungi, plants, animals or humans.

In particular, the nucleic acids or parts thereof according to the invention are inserted into a vector, a plasmid, a cosmid, a chromosome or minichromosome, a transposon, an IS element, an rDNA, or all kinds of circular or linear DNA or RNA.

The skilled person will understand that nucleic acids having at least 4⁰% homology with the nucleic acids according to the invention can also be employed according to the invention. The proteins can also be modified without losing their function. Thus, for example, so-called conservative exchanges of amino acids may be performed. For example, hydrophobic amino acids or hydrophilic amino acids can be interchanged.

For the preparation, isolation and characterization of the ciliate major secreted proteins and for determining the sequences of such proteins, the following methods can be used.

Two-dimensional gel electrophoresis of cell-free supernatants of a Tetrahymena culture

For obtaining Tetrahymena supernatants from cultures grown on PPYS medium, the following procedure was employed:
5×400 ml each of PPYS in a Fernbach flask was inoculated with 50,000 is cells/ml of the strain B1868.7 and subsequently incubated at 30° C. and with 80 rpm on an Infors shaker. The harvesting of the cells was performed in the following way and with the following result:

Harvest of the cells, in oil test beakers:
Cell titer: 1,000,000 cells/ml
Cell mass: 2 ml/100 ml of medium

From 2 l of harvest, 1930 ml of supernatant was obtained, which was concentrated to 70 ml through a Pellicon XL unit (Millipore).

The two-dimensional gel electrophoresis was performed in the following way:

Precipitation of 200 μl of concentrated PPYS supernatant with trichloro acetic acid (TCA):
200 μl supernatant, with TCA (50%; w/v) ad 1000 μl
10 min on ice
5 min, 20,000×g, 4° C.
decanting off the TCA
3× washing the pellet with ice-cold diethyl ether and centrifugation as above
drying the pellet, followed by uptake in 270 μl of Sanchez buffer

Then, 270 μl of sample volume was completely added to the reaction chamber (Biorad Protean IEF Cell), covered with Amersham IPG (13 cm, pH 4-7), and overlaid with about 800 μl of paraffin.

Program Cycle in the Isoelectric Focusing System of the BIORAD COMPANY:

| | |
|---|---|
| active rehydration | 50 V, 12 h |
| cooling to 20° C. | |
| focusing (1st dimension) | 300 V, 1 mA, 4 h |
| | 1900 V, 1 mA, 5 h |
| | 3500 V, 1 mA, 7 h |

After the program was completed, the IPGs were transferred into glass tubes and stored at −20° C. until further use.

The SDS Gel Electrophoresis (2nd Dimension) was Performed as Follows:

equilibration of the IPGs (2× in 12.5 ml of equilibration solution)
casting of 12% SDS gels with only one large sample pocket plus marker pocket
inserting the IPGs into the large sample pocket; for this purpose, the IPGs had to be shortened (pH 4 directly beside the protein marker)
run at 200 V
Coomassie blue staining (analytical)
decoloring over night (analytical)

The Coomassie-stained gels were sealed by welding, and the peptide sequences were established.

For establishing the N-terminus of the selected proteins, it was necessary to blot the proteins from the two-dimensional gel electrophoresis onto PVDF membranes.

This was effected in accordance with the manual “Immobilon-P Transfer Membrane User Guide” from the Millipore company.

Molecular-biological examination of the ciliate major secreted proteins using the CMSP 0 protein as an example

After the purity of the protein had been demonstrated, samples of the protein were blotted onto a PVDF membrane as already described above and subjected to initial sequencing from the N terminus. In addition, a further sample was tryptically digested and also subjected to initial sequencing. Using the protein sequences obtained thereby, oligonucleotide primers were prepared, which were then employed in reverse transcriptase PCR (3′ RACE, rapid amplification of cDNA ends). Using this PCR technique, cDNA of CMSP 0 was successfully amplified and subsequently sequenced. The sequence obtained had a length of 630 bases. In the sequence derived, the oligonucleotides of 13 and 15 amino acids established in the internal protein sequencing were found again to 100%. Using the Universal Genome Walker™ kit of the company Clontech Laboratories (Palo Alto, USA), the sequences of the N terminus of the mature protein, that of the pre/pro peptide as well as the upstream and downstream sequences could be established. The peptide sequence of the N terminus also corresponded to the sequence already established. The pre/pro peptide is a peptide having a length of 116 amino acids which bears both the signal sequence and the pro peptide. Sequence comparisons yielded high homologies with cysteine proteases of a wide variety of organisms. In addition to the downstream region known from the 3′RACE, another 302 bases could be established. Upstream, a region of 1112 bases was edited.

The invention also relates to the protein CMSP 1 (Seq. ID. No. 9) of amino acids 1 to 119, the related pre/pro peptide of amino acids 120 to 324 (Seq. ID. No. 8), and the DNA coding for it with the non-coding 5′ and 3′ regions according to Seq. ID. No. 7. The nucleic acid sequence of the non-translated region (upstream region) upstream from the coding sequence region of CMSP 1 from Tetrahymena is found between position −365 and position −1 (represented in lowercase letters). It is a subject matter of the invention as the regulatory element of Seq. ID. No. 10. The coding sequence region of the cDNA is represented in capital letters. With the start codon ATG, the numbering of the sequence begins. Regions known from the protein sequencing are printed in boldface, and the stop codon is underlined. The mature protein is coded from base 358. The sequence protocol from base 1 to base 357 represents the pre/pro sequence of CMSP 1. The sequence protocol from base 358 to base 975 represents the sequence of the mature protein. In position 973, there is the translation stop TGA. The nucleic acid sequence from position 976 to position 1052, which is below the coding sequence of the protein CMSP 1 from Tetrahymena, is the downstream region of the protein, which is not translated (also represented in lowercase letters).

```
Seq. ID. No. 1
ATG AGA ACT CAA TTG CTT ATT GCT GCT GCT TTA GGT
TTA ACC TTA TTA
GGT TTA ACT TCC TAT TTA TTC CTC CAC AAG TCT ACT
CAA GTT GGA TAC
ACT GAT GAC TAA ATT AAC ATG TGG AAG GGC TTC AAG
AAG ACC TAC AAC
AAA AAA TTC TCT TCT GAA GAT GCT GAC TAA GAA GCT
TAC AGA ATG AAC
GTC TTC TTC GAT AAC GTT GAA TAC GCT TCA TAA GAT
TCT ACC ATG GGT
ATT ACC AAG TTT ATG GAC CTT ACC CCT GTT GAA TTT
GCC TAA CTT TAC
TTG AAT CCC ATT GAA AAC GTT GAA GGT TCT ATT GAA
ACT TTC TAA GCT
ATT CAA GCT AAT GGA GAT ATT GTT GTC GAT TGG GTT
GCT AAG GGT
GCT GTC ACA CCT GTT AAG GAT CAA GGT GGT TGT GGT
GGT TGT TGG
TCT TTC GCT ACT ACT GGT GGT GTT GAA GGT GCT AAC
TTT GTC TAC AAA
AAT GTC CTC CCT AAC TTA TCT GAA CAA TAA TTA ATC
GAC TGT GAC ACT
TAA AAC AGT GGT TGC GGT GGT GGT TTA AGA GAC GTT
GCC TTA AAC TAC
GTT AAG GCA ACT GGT TTG GCC ACT GAA TAA GAT TAT
CCT TAT GAA
GCT AAG GAT GGT AAG TGC AGA CTT GAA GGT AAG AGC
CAC CCT TGG ACT
GTT TCT GGT TAC ACT TCT ATT AAG TAA TGC GCT GAC
CTC GTT ACT GCT
ATT CAA AAG GCC CCT GTT ACC GTT GGT ATC GAT GCT
TCT AAC CTC TAA
TTC TAC ACT GGT GGT ATC TTT TCT AAG TGC GCC ACT
AAC ATC AAC CAC
GGT GTT TTA CTT GTT GGT TAC GAC TCT GTT AAT CAA
TCT TGG AAG GTC
AAG AAC TCC TGG GGT CCT AAC TTC GGT GAA CAC GGT
TAC TTC TAA CTC
TCT GCT AAG GTT ACT GGT GAC CAA ATT GCT AAC ACT
TGC GGT ATT
TGC TCT AGA GCT TAT GCT CCT TAC ATT TGA
Seq. ID. No. 2
M R T Q L L I A A A L G L
T L
L G L T S Y L F L H K S T
Q V
G Y T D D Q I N M W K G F
K K
T Y N K K F S S E D A D Q
E A
Y R M N V F F D N V E Y A
S Q
D S T M G I T K F M D L T
P V
E F A Q L Y L N P I E N V
E G
S I E T F Q A I Q A N G D
I V
V D W V A K G A V T P V K
D Q G
G C G G C W S F A T G G V
E G
A N F V Y K N V P N L S E
Q Q
L I D C D T Q S G C G G G
L R
D V A L N Y K A T G L A T
E Q
D Y P Y E K D G K C R L E
G K
S H P W V S G Y T S I K Q
C A
D L V A I Q K A P V T V G
I D
A S L Q F Y T G G I F S K
C A
T I N H G V L L V G Y D S
V N
S W K V K N S W G P N F G
E G
Y F Q L S A K V T G D Q I
N T
C G I C S R A Y A P Y I
Seq. ID. No. 3
                                                                                aaaa
aagtttaccc
attgttccg attttatatt taaaaattaa agaaagaatt
aaattgaatc
tttttttctt ttataaaatc tataaaagat tgagataaca
aaaagttgat
aaaaatataa aatattcata catttaattt aagcatatta
aaatacattt
catagttgaa aaataaagaa aacagtatct ataaaaacta
tgctgaaagt
ttaatgctga aagtttactt ctcgttttta tttaactttt
ctagtttaaa
ataattatta atatgaattg aaataattgt tactattagc
ttttattgaa
ttcaatttat taaaaattgg atctaatgta atcttagaaa
taataactaa
aaatgggatt tgaaaaatct agataagaat ataaaattaa
aaatcaaatt
aatcgaatct tttattacat ctcattaaaa agttaataaa
ataaaaaata
aagttttatt cttattttaa tatcattttt taaattacca
taatcaattt
taacttaaag cttcaataaa aaaatatata ttttagaaac
tttaataaac
tattgagcaa cttataaaga attaaaaata tttttcattt
gtaaaatgaa
atgaaaaata ttatcctgag cttacaaatc ttttataaac
atttttttat
ttataatttt cttttatatt aaggttttct aatatcgaat
aagatttctg
ctcagagaaa attttctgca atttaataaa taataaaaga
atttttatgt
aaaagaatta atttaatcta gaccttagaa aatgaatgaa
tcaatatata
cttttaactc tgttttgcat gagtaaacaa atgagttttt
tacatcaaaa
cagtttttac attttgtatg taatcaaaaa agctttactt
tgactaaaat
attgaaagat ttgctttaaa gctaaaatat acattaaact
attaaagatt
tttatttata attctttcat aataaatcat taacaaataa
acaaacaaac
aaagaaaaaa attatttagt caagctttaa aaaattatta
attgaaataa
tatttgatat aattaaatta atctaaaaa acataagata
gaataaaata
Seq. ID. No. 4
aaa atc ttt caa aaa tat ata aaa tga tta ata taa
aac ttt ata ttt tta tag taa tta ata
tta aaa act ttt tgt tta act att tat agg aaa taa
tat tat tta cta taa aca agc cag aca
act att ctt ttg ttt tta tta ctt ttt tat aca aaa
aac tga taa caa atc aat tca aat aaa
tat tct att atc atg aaa gtc tta cat ttt att tta
aca aaa aat aaa aag aat cta ttt ttt
aat ttt gac ttg aat tac aac ttt tat aac aaa tca
aac ttt aac aat tta taa tta aaa att
tta atc cac taa tta att gac tga ata t
Seq. ID. No. 5
M R T Q L L I A A A L G L
T L
L G L T S Y L F L H K S T
Q V
G Y T D D Q I N M W K G F
K K
T Y N K K F S S E D A D Q
E A
Y R M N V F F D N V E Y A
S Q
D S T M G I T K F M D L T
P V
E F A Q L Y L N P I E N V
E G
S I E T F Q A I Q A N
Seq. ID. No. 6
G D I V V D W V A K G A V
T P
V K D Q G G C G G C W S F
A
T G G V E G A N F V Y K N
V
P N L S E Q Q L I D C D T
Q
S G C G G G L R D V A L N
Y
K A T G L A T E Q D Y P Y
E
K D G K C R L E G K S H P
W
V S G Y T S I K Q C A D L
V
A I Q K A P V T V G I D A
S
L Q F Y T G G I F S K C A
T
I N H G V L L V G Y D S V
N
S W K V K N S W G P N F G
E
G Y F Q L S A K V T G D Q
I
N T C G I C S R A Y A P Y
I
Seq. ID. No. 7:
    cttaacag agagtatcct gtaaattaaa agttattaac
attccccact
cgatcaattt cttttacact ctttaaacga aatgtttcga
ttttatccat
caaaattctt aattcttata tatttttaac tattaaaata
tcagattgat
aattcatgcc atttgtggtg ttttaaatac aataatacgt
atatcatctt
gcataacttt caaagccaca aaaactaatt gatcctattt
ttatagatta
ggaaactatt tcttttatat tttgtagaca tctaattatt
actttaaata
ctagattatc taatacatcc tttcataaaa gattcaatta
aataaattta
aactaaacaa aaaaaatATG AGCTAAAAAA TTACTGTTAC
TCTTGTTGCT
ATCGCTGCTA TTGCCGCTAT CACTGCTGCT GGCATTTACT
ACTAGAACCA
CTAAGCTAGC CAATTAGAAA AGTCTTTCAA GAGAAATACC
ATCCTTGAAT
AATGGAACGA ATTCAAGCAA AAGTTTGGTA AGAAATATGC
TGACTAAGAA
TTCGAAAGAT ACAGAATCGG AGTTTTCGCT CAAAATTTAG
AAGTTATCAA
GAACGATCCT TCCTTCGGTG TTACCAAGTT CATGGATATG
ACCCCATAAG
AATTCGAACA ATCCTACTTA TCTCTCTAAC TCCAACAAAA
CTTCAATGCT
GAAAAGGTTG ATGGTGACTT TAATGGTGAT ATTGATTGGA
CTTAAAAGGG
TGCTGTTACT CCTGTCAAGG ACTAAGGTTC TTGCGGTTCT
TGCTGGGCTT
TCTCTGCTAT CGGAGCTGTT GAATCTGCTT TGATCTTGAA
CGGTGAAGAC
AAGAACATCA ATTTGGCTGA ATAAGAATTG GTCGACTGTG
CTACTACTCC
CAAGTACGAA AATGAAGGTT GCAACGGTGG TTGGATGGAC
TCTGCTTTCG
ACTACATTAT TGATGAAAAG ATCTCTTAAA CCAAGGACTA
CAAGTACACT
GCTAGAGACG GCAAGTGCAA GGATACCTCA TCTTTTGAAA
AGAAGTCTAT
CTCTGGATAC AAGGACATTC CTCAAGGTGA CTGCAAGTCT
CTCTTAAACG
CTCTTTCCTA ATAACCCGTT GCTATTGCAG TTGATGCTTC
TTCTTGGTAA
TTCTACAACA AGGGAGTTTT ATCATCCTGT GGCAGCAGAC
TTAATCACGG
TGTTTTATTA ACTGGTTACG TTAACGAAAC TTACAAGGTT
AAGAACTCTT
GGGGTACTTC TTGGGGTGAA AAGGGTTTCA TCTAATTAAA
GTCAGGTAAC
TCTTGTGGTC TCTGCAATGC TGCCTCTTAC CCTCTTGCTT
GAaaaaaata
cttaaataat taaaaaaaat agtattatta tataatccat
attaaagtct
ttttttataa atctttaaa
Seq. ID. No. 8:
M S Q K I T V T L V A I A A I A A I T A A G I Y Y
Q N H Q A S Q L E K S F K R N
T I L E Q W N E F K Q K F G K K Y A D Q E F E R Y
R I G V F A Q N L E V I K N
D P S F G V T K F M D M T P Q E F E Q S Y L S L Q
L Q Q N F N A E K V D G D
F N
Seq. ID. No. 9:
G D I D W T Q K G A V T P V K D Q G S C G S C W A
F S A I G A V E S A L I L
N G E D K N I N L A E Q E L V D C A T T P K Y E N
E G C N G G W M D S A F D
Y I I D E K I S Q T K D Y K Y T A R D G K C K D T
S S F E K K S I S G Y K D I P
Q G D C K S L L N A L S Q Q P V A I A V D A S S W
Q F Y N K G V L S S C G S
R L N H G V L L T G Y V N E T Y K V K N S W G T S
W G E K G F I Q L K S G N
S C G L C N A A S Y P L A
Seq. ID. No. 10:
    cttaacag agagtatcct gtaaattaaa agttattaac
attccccact
cgatcaattt cttttacact ctttaaacga aatgtttcga
ttttatccat
caaaattctt aattcttata tatttttaac tattaaaata
tcagattgat
aattcatgcc atttgtggtg ttttaaatac aataatacgt
atatcatctt
gcataacttt caaagccaca aaaactaatt gatcctattt
ttatagatta
ggaaactatt tcttttatat tttgtagaca tctaattatt
actttaaata
ctagattatc taatacatcc tttcataaaa gattcaatta
aataaattta
aactaaacaa aaaaaat
```

1. A regulatory element of a DNA for an efficient heterologous expression of proteins in Tetrahymena ssp which efficient heterologous expression is performed under control of promoters and/or terminators which are derived from in Tetrahymena ssp naturally occurring DNA comprising promotors and/or terminators and a coding region for proteins secreted ion a high level and the expression of proteins secreted on a high level is independent of the cell-cycle of Tetrahymena ssp.

2. The regulatory element of a DNA of claim 1 wherein the DNA is obtainable by
isolating proteins of Tetrahymena ssp which proteins are secreted on a high level into a fermentation broth and
the secretion of the proteins is occurring on a high level independently of the cell-cycle of Tetrahymena ssp,
determination of at least one partial amino acid sequence of the proteins;
establishing the nucleic acid sequence of the proteins and there from establishing the gene which codes for these proteins;
establishing the regulatory elements of the coding region of said proteins by methods known as such in molecular biology.

3. The regulatory element of claim 1, obtainable from Tetrahymena ssp using gene constructs made from regulatory elements selected from the group consisting of promoters or terminators from Tetrahymena and coding nucleic acid sequences of a protein to be expressed heterologously, said regulatory elements from Tetrahymena being obtainable by:
two-dimensional gel electrophoretical separation and isolation of the proteins (CMSP) selected from the group consisting of:

| | Molecular | Isoelectric |
|---|---|---|
| | weight | point |
| CMSP | ±0.5 kD | ±0.2 units |
| No. | (kD) | (pH units) |
| 0 | 22.22 | 5.9 |
| 1 | 24.96 | 7.5 |
| 2 | 16.04 | 7.3 |
| 3 | 24.96 | 6.8 |
| 4 | 23.76 | 6.5 |
| 5 | 23.76 | 5.5 |
| 6 | 30.38 | 6.8 |
| 7 | 31.91 | 6.8 |
| 8 | 25 | 8.5 |
| 9 | 16.44 | 6.9 |
| 10 | 11.2 | 5.5 |
| 11 | 37.9 | 7.2 |
| 12 | 37.9 | 7.8 |
| 13 | 21.54 | 7.4 |
| 14 | 24.36 | 8.7 |
| 15 | 14.4 | 4.7 |
| 16 | 14.4 | 4.9 |
| 17 | 19.05 | 6.4 |
| 18 | 22.6 | 7.2 |
| 19 | 20.01 | 7.4 |
| 20 | 29.6 | 7.3 |
| 21 | 30 | 7.3 |
| 22 | 30.38 | 7.5 |

determination of at least one partial amino acid sequence of the proteins;
establishing the nucleic acid sequence of the proteins and herefrom establishing the gene which codes for these proteins;
establishing the regulatory elements of the coding region of said proteins.

4. The regulatory element according to claim 3 having the Seq. ID. No. 3, 4, 8 or 10.

5. A method for heterologous expression of proteins in Tetrahymena ssp in a broth which proteins are secreted on a high level into the broth by employing a regulatory element of claim 1.

6. The method for the heterologous expression of proteins from Tetrahymena according to claim 5 using gene constructs made from regulatory elements selected from the group consisting of promoters or terminators from Tetrahymena and coding nucleic acid sequences of a protein to be expressed heterologously, said regulatory elements from Tetrahymena being obtainable by:
two-dimensional gel electrophoretical separation and isolation of the proteins (CMSP) selected from the group consisting of:

| | | Isoelectric |
|---|---|---|
| | Molecular weight | point |
| | ±0.5 kD | ±0.2 units |
| CMSP No. | (kD) | (pH units) |
| 0 | 22.22 | 5.9 |
| 1 | 24.96 | 7.5 |
| 2 | 16.04 | 7.3 |
| 3 | 24.96 | 6.8 |
| 4 | 23.76 | 6.5 |
| 5 | 23.76 | 5.5 |
| 6 | 30.38 | 6.8 |
| 7 | 31.91 | 6.8 |
| 8 | 25 | 8.5 |
| 9 | 16.44 | 6.9 |
| 10 | 11.2 | 5.5 |
| 11 | 37.9 | 7.2 |
| 12 | 37.9 | 7.8 |
| 13 | 21.54 | 7.4 |
| 14 | 24.36 | 8.7 |
| 15 | 14.4 | 4.7 |
| 16 | 14.4 | 4.9 |
| 17 | 19.05 | 6.4 |
| 18 | 22.6 | 7.2 |
| 19 | 20.01 | 7.4 |
| 20 | 29.6 | 7.3 |
| 21 | 30 | 7.3 |
| 22 | 30.38 | 7.5 |

determination of at least one partial amino acid sequence of the proteins;
establishing the nucleic acid sequence of the proteins and therefrom establishing the gene which codes for these proteins;
establishing the regulatory elements of the coding region of said proteins.

7. Proteins obtainable from Tetrahymena thermophila having the following properties:

| | | Isoelectric |
|---|---|---|
| | Molecular weight | point |
| | ±0.5 kD | ±0.2 units |
| CMSP No. | (kD) | (pH units) |
| 0 | 22.22 | 5.9 |
| 1 | 24.96 | 7.5 |
| 2 | 16.04 | 7.3 |
| 3 | 24.96 | 6.8 |
| 4 | 23.76 | 6.5 |
| 5 | 23.76 | 5.5 |
| 6 | 30.38 | 6.8 |
| 7 | 31.91 | 6.8 |
| 8 | 25 | 8.5 |
| 9 | 16.44 | 6.9 |
| 10 | 11.2 | 5.5 |
| 11 | 37.9 | 7.2 |
| 12 | 37.9 | 7.8 |
| 13 | 21.54 | 7.4 |
| 14 | 24.36 | 8.7 |
| 15 | 14.4 | 4.7 |
| 16 | 14.4 | 4.9 |
| 17 | 19.05 | 6.4 |
| 18 | 22.6 | 7.2 |
| 19 | 20.01 | 7.4 |
| 20 | 29.6 | 7.3 |
| 21 | 30 | 7.3 |
| 22 | 30.38 | 7.5. |

8. Proteins having the Seq. ID. No. 2, 5, 6, 8 or 9.

9. A nucleic acid coding for the protein according to claim 7, especially having the nucleic acid sequence Seq. ID. No. 1, 3, 4, 7, and combinations thereof.
